(19) United States
(12) Patent Application Publication
(10) Pub. No.: US 2022/0415465 A1
(43) Pub. Date: Dec. 29, 2022

(54) SELF-BENCHMARKING FOR DOSE GUIDANCE ALGORITHMS

(71) Applicant: Novo Nordisk A/S, Bagsvaerd (DK)

(72) Inventors: Henrik Bengtsson, Taastrup (DK); Tinna Bjoerk Aradottir, Copenhagen (DK); Zeinab Mahmoudi, Birkeroed (DK); Ali Mohebbi, Soeborg (DK); Julia Rosemary Thorpe, Gentofte (DK)

(21) Appl. No.: 17/776,146

(22) PCT Filed: Dec. 3, 2020
(86) PCT No.: PCT/EP2020/084440

§ 371 Date: May 11, 2022

(30) Foreign Application Priority Data
Dec. 3, 2019 (EP) 19213259.5

(51) Int. Cl. G16H 20/10; G16H 10/60; A61B 5/145; A61B 5/00

(52) U.S. Cl. CPC G16H 20/10; G16H 10/60; A61B 5/14532; A61B 5/4848; A61B 5/742

(57) ABSTRACT

A benchmarking approach is employed that compares advice output from one or more alternative treatment guidance algorithms with a current actual treatment in terms of treatment outcomes. Treatment outcome for the current strategy is reflected in an actual BG outcome or profiled. Treatment outcome for an alternative algorithm-generated dose advice is based on a patient-specific model. The two sets of outcomes can be compared directly or using performance scores as a weighted combination that penalises or rewards certain outcomes. A statistical test may be applied to the accumulated results (paired outcomes or scores) to determine whether the algorithm is superior to the user's current dosing strategy, or alternative strategies.

The present disclosure generally relates to systems and methods for assisting patients and health care practitioners in managing insulin treatment to diabetics. In a specific aspect the present invention relates to systems and methods suitable for use in a diabetes management system that helps to identify a best-performing and most suitable dose recommendation algo-rithm/strategy between one or more alternatives.

### BACKGROUND

Diabetes mellitus (DM) is impaired insulin secretion and variable degrees of peripheral insulin resistance leading to hyperglycaemia. Type 2 diabetes mellitus is characterized by progressive disruption of normal physiologic insulin secretion. In healthy individuals, basal insulin secretion by pancreatic β cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours. Years of poorly controlled hyperglycaemia can lead to multiple health complications. Diabetes mellitus is one of the major causes of premature morbidity and mortality throughout the world.

Effective control of blood/plasma glucose can prevent or delay many of these complications but may not reverse them once established. Hence, achieving good glycaemic control in efforts to prevent diabetes complications is the primary goal in the treatment of type 1 and type 2 diabetes. Smart titrators with adjustable step size and physiological parameter estimation and pre-defined fasting blood glucose target values have been developed to administer insulin me-dicament treatment regimens.

There are numerous non-insulin treatment options for diabetes, however, as the disease pro-gresses, the most robust response will usually be with insulin. In particular, since diabetes is associated with progressive β-cell loss many patients, especially those with long-standing disease will eventually need to be transitioned to insulin since the degree of hyperglycemia (e.g., HbA1c≥8.5%) makes it unlikely that another drug will be of sufficient benefit.

The ideal insulin regimen aims to mimic the physiological profile of insulin secretion as closely as possible. There are two major components in the insulin profile: a continuous basal secretion and prandial surge after meals. The basal secretion controls overnight and fasting glucose while the prandial surges control postprandial hyperglycemia.

Based on the time of onset and duration of their actions, injectable formulations can be broadly divided into basal (long-acting analogues [e.g., insulin detemir and insulin glargine] and ultra-long-acting analogues [e.g., insulin degludec]) and intermediate-acting insulin [e.g., isophane insulin] and prandial (rapid-acting analogues [e.g., insulin aspart, insulin glulisine and insulin lispro]). Premixed insulin formulations incorporate both basal and prandial insulin components.

There are various recommended insulin regimes, such as (1) multiple injection regimen: rapid-acting insulin before meals with long-acting insulin once or twice daily, (2) premixed analogues or human premixed insulin once or twice daily before meals, and (3) intermediate- or long-acting insulin once or twice daily.

Algorithms can be used to generate recommended insulin dose and treatment advice for diabetes patients. However, for a given patient a number of relevant dose recommendation algorithms may be relevant and choosing the one providing the best guidance may be a challenge.

Correspondingly, it is an object of the present invention to provide systems and methods suitable for use in a diabetes management system that helps to identify the best-performing and most suitable dose recommendation algorithm between a number of alternatives.

However, the quality of advice provided by such algorithms depends on many factors that are difficult to control in a real-world setting. These include the user's individual profile, behaviour, adherence, and variance in parameters such as fasting blood glucose (FBG), glucose profile indicator (GPI) or ambulatory glucose profile (AGP). Quality of data inputs further affects algo-rithm quality, for example, glucose data depends on accuracy and correct use of a blood glucose monitor (BGM) or continuous glucose monitor (CGM).

This imperfect nature of real-world data, treatment adherence, device use, and other inevitable disturbances all degrade algorithm quality, such that the treatment advice provided may not be correct which makes it difficult to evaluate and benchmark the performance of alternative dose recommendation algorithms.

Having regard to the above, it is a further object of the present invention to provide systems and methods which take into consideration the nature of real-world data having been influ-enced by the many factors that are difficult to control and quantify in a real-world setting.

### DISCLOSURE OF THE INVENTION

In the disclosure of the present invention, embodiments and aspects will be described which will address one or more of the above objects or which will address objects apparent from the below disclosure as well as from the description of exemplary embodiments.

In summary, the proposed solution to the problem is to employ a benchmarking approach that compares advice output from any treatment guidance algorithm with the current actual treatment in terms of treatment outcomes. Treatment outcomes may be calculated for the user's actual dose based on their glucose profile following insulin intake, and for algorithm-generated dose advice based on an alternate profile estimated using the actual glucose profile, change in dose, and a patient-specific model. The two sets of outcomes may be compared directly or using performance scores as a weighted combination that penalises or rewards certain outcomes. A statistical test may be applied to the accumulated results (paired outcomes or scores) to determine whether the algorithm is superior to the user's current dosing strategy, or alternative strategies.

The self-benchmarking algorithm relies on two key data inputs: insulin dose and glucose level. The user's actual dose can be manually input or recorded automatically using a connected drug delivery pen or pen attachment to capture dose data. Devices for CGM provide data describing glucose level, including following intake of the insulin dose. This information, together with a known dose generated by any treatment guidance algorithm, can be used to retrospectively estimate the impact of the change in dose (from actual to advised) on the glucose response, and thus an alternate set of treatment outcomes. Additional information regarding context, lifestyle or behavioural factors may further be gathered from connected devices or sensors (e.g. mobile phone, wearable biosensors) to label results, such that an algo-rithm's performance can be evaluated both overall and for certain conditions (e.g. a specific time of day, level of physical activity, meal size etc.).

With this approach an alternative algorithm is only enabled to send advice to users once its superiority to the user's current treatment is demonstrated to be robust. The algorithm therefore only performs when it can perform well, leading to safer and more efficacious treatment advice.

Thus, in a first aspect of the invention a computing system for providing medication dose guidance recommendations for a query subject (patient) to treat diabetes mellitus is provided. The system comprises one or more processors and a memory in which is stored instructions that, when executed by the one or more processors, perform a method of evaluating and bench-marking one or more alternative dose guidance algorithms (DGAs) against a current DGA.

The instructions comprise the steps of obtaining a first data set and a second data set. The first data set comprises a plurality of glucose measurements of the query subject taken over a time course and thereby establishes a blood glucose history (BGH), each respective glucose measurement in the plurality of glucose measurements comprising (i) a blood glucose (BG) value and (ii) a corresponding blood glucose timestamp representing when in the time course the respective glucose measurement was made. The second data set comprises an insulin dose event history (IH) of the query subject, wherein the IH comprises at least one dose event during all or a portion of the time course, each dose event of the at least one dose event comprising (i) a dose amount and (ii) a corresponding dose event timestamp representing when in the time course the respective dose event occurred.

The instructions comprise the further steps of obtaining a current DGA, one or more alternative DGAs adapted to calculate an alternative dose recommendation based at least on BGH, and a physiological model (PM) for the query subject adapted for modelling a BG response based on BGH and an amount of insulin injected at a given time. Alternatively, utilizing more advanced DGAs also IH data may be utilized when calculating dose recommendations.

Corresponding to a recent dose event, e.g. the most-recent, performed in accordance with the current dose strategy, for a given alternative DGA the instructions comprise the further steps of (i) determining an alternative dose recommendation, (ii) utilizing the PM to calculate an alternative BG treatment outcome, (iii) and comparing and benchmarking the alternative BG treatment outcome against the measured BG treatment outcome. If the benchmarking for the given DGA exceeds a given set of benchmarking criteria, the instructions comprise the further step of suggesting or implementing the given alternative DGA to substitute the current DGA. The former current DGA may then become a new alternative DGA.

In this way, once a given dose guidance tool demonstrate superiority over a current strategy, the best performing tool can be selected and enabled either automatically by the benchmarking algorithm, or by the user based on feedback regarding performance.

It should be noted that knowledge of the actual current strategy is not essential for the performance of the present invention—it could even be a ‘no strategy’ in which the patient just takes a fixed bolus each morning. Correspondingly, in the context of the present invention the term “current DGA” should be understood to also cover such simple strategies which per se hardly can be characterized as an algorithm. Indeed, once such a simple initial “strategy” has been replaced by a better-performing DGA the current DGA will be a “real” DGA. However, as for the initial simple strategy, knowledge of the current DGA is not essential to the performance of the present invention.

The instructions may comprise the step of obtaining a current DGA and may comprise the further step of determining a current dose recommendation utilizing the current DGA. The current DGA may be adapted to calculate a dose recommendation based at least on BGH.

The term “treatment outcome” indicates that the subsequent BG outcome is expected to reflect that the recommended dose is actually injected by the patient, i.e. that a “dose event” repre-sents an injection event.

Comparing the outcome from the current and the one or more alternative dose recommendation algorithms will typically be to determine how the BG outcome (real or calculated) performs in relation to a given treatment target for the patient and then benchmark the results. For a bolus dose of a fast-acting insulin the BG outcome will in most cases reflect the patient's BG after a meal and the treatment target will typically be a desired BG range. The BG outcome may be in the form of a simple BG value representing e.g. a maximum (or minimum) BG value measured/calculated within a given period after a meal, or it may be in the form of an area for a curve portion. In a simple form the BG outcome is represented by a single BG value deter-mined/calculated for a given point in time after a meal. Alternatively, a BG outcome may be determined by continuous (or quasi continuous) BG measurement (e.g. by a skin mounted CGM device) and a corresponding calculated outcome profile for the alternatives, this allowing both maximum/minimum values to be determined as well as curve analysis to be performed.

Just as a BG meter or a CGM device may allow the system to obtain BG values automatically via wireless transmission of data to a main computing unit such as a smartphone, also dose event data may be obtained automatically by a drug delivery device provided with dose logging functionality.

The benchmarking may incorporate different aspects of the outcomes, e.g. the maximum and minimum BG values determined/calculated or the time in which the patient is outside of within the treatment target range. Some outcomes may be over-weighted as less desirable, e.g. BG values below the target range.

For each alternative DGA the step of comparing and benchmarking may be performed for a plurality of alternative BG treatment outcomes against the corresponding measured BG treatment outcomes for a given period of time, e.g. corresponding to all dose events for a given period such as the most-recent weeks or months, e.g. the last 2 weeks or the last month.

The resulting historical dataset can be used to apply a statistical test (e.g. ratio t-test) comparing the user's current dose strategy with each alternative. Once the dataset is large enough, statistically significant superiority of any algorithm over the user's current strategy will be reflected in the results of the statistical test, e.g. a significant p-value for the ratio t-test.

The step of comparing and benchmarking may be performed for a plurality of alternative BG treatment outcomes in accordance with an identifier representing specific contextual conditions allowing the benchmarking to filter results based on specified conditions, e.g. type of meal, period of the day, periods with activity or periods with sickness. The identifiers may be entered manually by the patient or gathered automatically, e.g. temperature and heart rate reflecting exercise or sickness may be provided by body-worn devices such as a smartwatch. In this way alternative DGAs performing superiorly under certain contextual conditions can be identified and implemented.

In exemplary embodiments, for a given current dose recommendation, the instructions comprise the further steps of (i) utilizing the PM to calculate a calculated BG treatment outcome for the dose recommendation, and (ii) calculating a deviation BG outcome as the difference between the measured BG treatment outcome and the calculated BG treatment outcome. In this way it can be estimated to what extent all the unknown parameters not incorporated in the PM have contributed to the measured BG values, e.g. meals, behavior, habits, sickness, stress. For the corresponding alternative BG treatment outcome for a given alternative DGA, a corrected alternative BG treatment outcome can be calculated as the sum of the alternative BG treatment outcome and the deviation BG outcome, which then can be utilized in the comparing and benchmarking step, this providing a “level playing field” for the alternative DGAs.

In the above the steps of subtraction and addition are disclosed in a given order, however, the disclosure covers that the steps may be performed in any order.

The comparing and benchmarking may typically be repeated and updated after each dose event.

In the above examples the DGAs are adapted for calculation of a bolus amount of fast-acting insulin, however, in a further aspect of the invention the DGAs are adapted for calculation of a dose recommendation for a long- or ultra-long-acting insulin. In such a set-up each DGA could be designed to provide a given level of aggressiveness in a dose titration regimen, this allowing a patient to reach and maintain the desired titration level faster and more efficient.

For a titration regimen the algorithm may be based on BG input in the form of values representing a titration glucose level value (TGL) which traditionally would be in the form of a fasting BG value taken manually by the patient in the morning. Alternatively, a TGL value may be determined based on CGM data. For example, a daily TGL may be determined as the lowest BG average for a sliding window of a predetermined amount of time, e.g. 60, 120 or 180 minutes, across the BG values for the corresponding day.

### BRIEF DESCRIPTION OF THE DRAWINGS

In the following embodiments of the invention will be described with reference to the drawings, wherein

FIG. 1 shows a flowchart of processes and features for a first embodiment of a system providing a dose guidance recommendation,

FIG. 2 illustrates how a plurality of alternative BG outcomes are calculated for a series of dose events,

FIG. 3 shows in diagrammatic form how a deviation analysis is used to calculate corrected alternative BG outcomes,

FIG. 4 illustrates how performance scores for alternative BG outcomes are statistically tested against BG outcome for a current dosing strategy,

FIGS. 5A and 5B show model output for an alternative algorithm respectively a current treatment strategy, and

FIGS. 6A and 6B show measured respectively simulated CGM time series for 4-hour postprandial intervals.

### DESCRIPTION OF EXEMPLARY EMBODIMENTS

Overall a diabetes dose guidance system is provided that helps people with diabetes by gen-erating recommended insulin doses. In such a system a given algorithm is used to generate recommended insulin doses and treatment advice for diabetes patients based on BG and insulin dosing history, however, many other factors will influence the BG outcome resulting from administration of a given dose of insulin. Correspondingly, a currently used algorithm for a given patient may not necessarily provide the best and most efficacious advice. As disclosed in greater detail above, the proposed solution to the problem is to employ a benchmarking approach that compares advice output from alternative treatment guidance algorithms with the current actual treatment in terms of treatment outcomes.

Essentially such a system comprises a back-end engine (“the engine”) which is the main aspect of the present invention used in combination with an interacting systems in the form of a client and an operating system.

The client from the engine's perspective is the software component that requests dose guidance. The client gathers the necessary data (e.g. CGM data, insulin dose data, patient parameters) and requests dose guidance from the engine. The client then receives the response from the engine.

On a small local scale the engine may run directly as an app on a given user's smartphone and thus be a self-contained application comprising both the client and the engine. Alternatively, the system setup may be designed to be implemented as a back-end engine adapted to be used as part of a cloud-based large-scale diabetes management system. Such a cloud-based system would allow the engine to always be up-to-date (in contrast to app-based systems running entirely on e.g. the patient's smartphone), would allow advanced methods such as machine learning and artificial intelligence to be implemented, and would allow data to be used in combination with other services in a greater “digital health” set-up. Such a cloud-based system ideally would handle a large amount of patient requests for dose recommendations.

Although a “complete” engine may be designed to be responsible for all computing aspects, it may be desirable to divide the engine into a local and a cloud version to allow the patient-near day-to-day part of the dose guidance system to run independently of any reliance upon cloud computing. For example, when the user via the client app makes a request for dose guidance the request is transmitted to the engine which will return a dose recommendation. Such a dose recommendation may correspond to what is calculated by the currently used algorithm or it may be calculated by an alternative algorithm having been enabled after a bench-marking analysis. In case cloud access is not available the client app would run a dose-recommendation calculation using the current algorithm. Dependent upon the user's app-settings the user may or may not be informed.

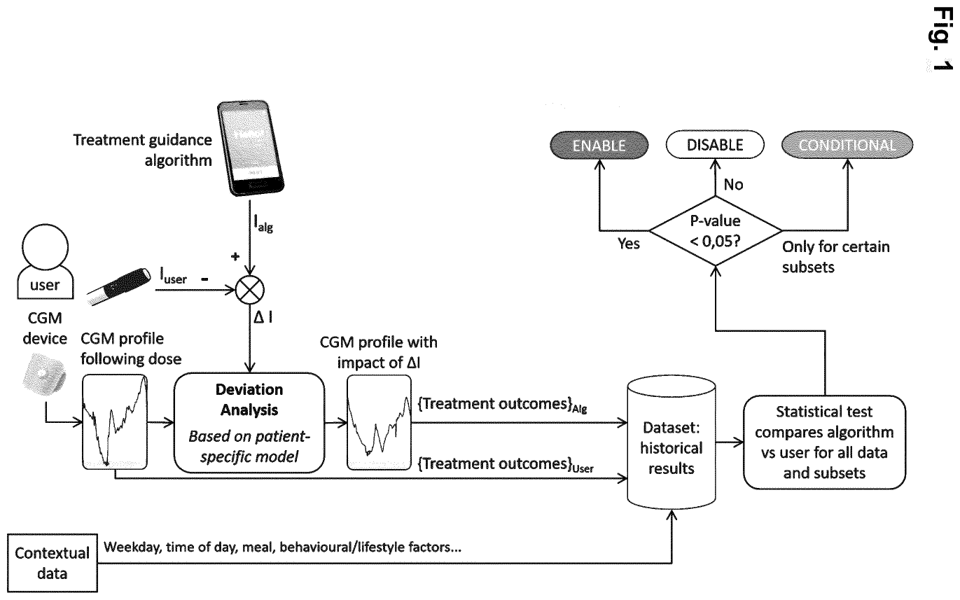

Turning to FIG. 1 an overview of a benchmarking process is shown. In the shown embodiment the system comprises a CGM device wirelessly transmitting a stream of BG data to the user's smartphone on which a client app is installed, as well as a pen drug delivery device with dose logging and data transmission capability, e.g. a Dialog® device mounted on a FlexTouch® pen, both provided by Novo Nordisk A/S, which wirelessly transmits dose event data to the user's smartphone. When a dose guidance request is made by the user, the app client will contact the engine (running on the phone or in the cloud) which returns a dose recommendation to be used by the user when setting and taking the next insulin dose using the drug delivery device. When a request is transmitted to the cloud engine all necessary data, e.g. BG data and dose logs for a given period may be transmitted with the request. Depending on the type of analysis performed during benchmarking, the period may be from a number of weeks to a number of months. Alternatively, historic data may be stored in the cloud and the app client will only transmit the latest not yet transmitted data.

When a user desires to take a dose amount of insulin, whether a basal or bolus type of insulin, he or she will start the app which will initially check that the most current data is available. The smartphone may be in continuous communication with the CGM device in which case BG data is automatically updated, however, in most cases (as for the Dialog® device) the app will prompt the user to manually activate the dose logging device to assure that the most recent dose event data is transmitted to the smartphone. In case data is not available the app may allow the user to enter data manually, e.g. a BG value determined by a strip-based BG meter. When data has been updated a dose guidance request may be transmitted to the engine (embedded in the app or in the cloud).

Before suggesting a new dose to the user, the system will perform a benchmarking of the currently running dose guidance algorithm (DGA) against the one or more alternative DGAs stored in memory. For a given past period, e.g. 4 weeks, for each dose event logged by the logging device (which is assumed to represent a dose injection) and for each alternative DGA an alternative dose recommendation is determined. Subsequently, using a physiological model (PM) for the patient adapted for modelling a BG response based on BG history (BGH data and an amount of insulin injected at a given time, an alternative BG treatment outcome profile is calculated.

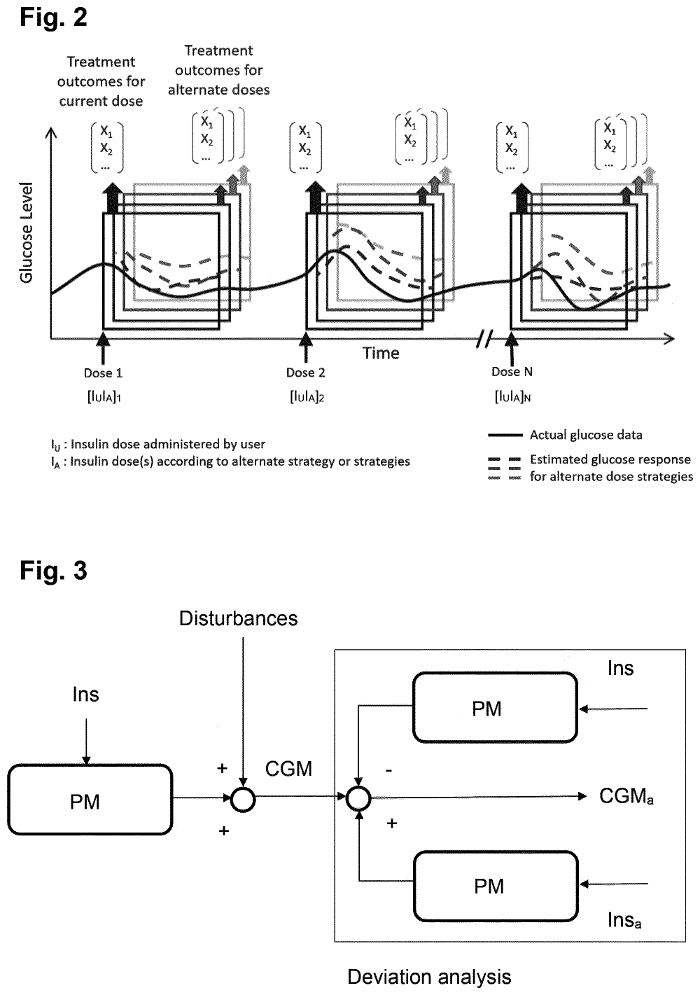

Additionally, for each dose event (i.e. assumed injected insulin amount) the PM is used to calculate an expected BG treatment outcome, this allowing the calculation of a deviation BG value as the difference between the measured BG treatment outcome and the expected BG treatment outcome. In this way it can be estimated to what extent all the unknown parameters (disturbances) not incorporated in the PM have contributed to the measured BG values, e.g. meals, behavior, habits, sickness, stress. Subsequently, for the corresponding alternative BG treatment outcome profile for a given alternative DGA, a corrected alternative BG treatment outcome profile can be calculated as the sum of the alternative BG treatment outcome and the deviation BG value, which then can be utilized in the comparing and benchmarking step, this providing a “level playing field” for the alternative DGAs (see FIG. 2).

More specifically, FIG. 3 illustrates how a realized and actually measured BG outcome (CGM) can be modelled as an insulin-based input determined by a physiological model (PM) with all other inputs influencing the BG outcome being categorized as “disturbances”, e.g., meals, stress, illness, physical activity, insulin model imperfection. In the deviation analysis the PM-based contribution from the current dose recommendation (Ins) is subtracted from the CGM outcome and the PM-based contribution from the alternative dose recommendation (Insₐ) is added to calculate a corrected alternative BG outcome (CGMₐ).

Just as historic BG and dose event data may have been stored in the app or cloud, also previously calculated corrected alternative BG treatment outcomes may have been stored such that these calculations only have to be performed for new events.

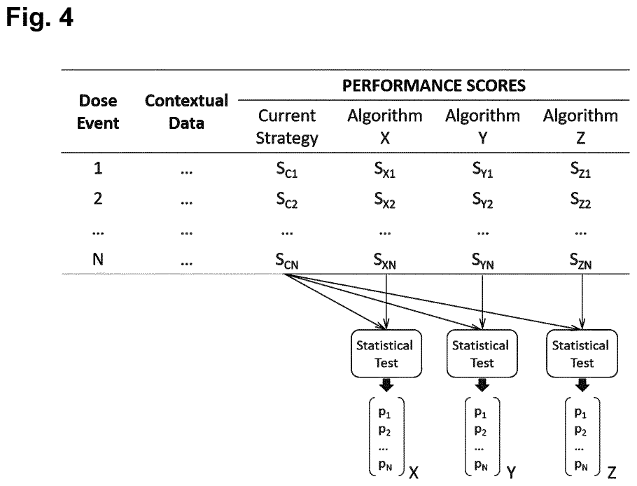

As a next step benchmarking and evaluation is performed by comparing performance, see FIG. 4. For each new dose event, treatment outcomes [X₁, X₂, . . . X_{M}] generated for each dosing strategy (current and all alternatives) are used to calculate a weighted performance score, S=λ₁X₁+λ₂X₂+ . . . +λ_{M}X_{M}, that penalises poor outcomes and/or rewards desirable outcomes. Contextual data (e.g. time of day, meal size, activity level) can also be stored for the dose event. The resulting historical dataset is used to apply a statistical test comparing the user's current dose strategy with each alternative. The comparison can either be for the full dose history or a subset thereof using contextual data to filter results based on specified conditions. Once the dataset is large enough, statistically significant superiority of any algorithm over the user's current strategy will be reflected in the results of the statistical test. For example, when the current treatment is compared with only one alternative algorithm ratio t-test may be used. If the current treatment is compared with multiple alternative algorithms an ANOVA test accompanied with post hoc multiple comparisons may be used

Once one or more DGAs demonstrate superiority over the user's current strategy, the best performing DGA is selected and enabled either automatically by the benchmarking algorithm, or by the user based on feedback regarding performance, this allowing the app to calculate and display a new recommended dose size as a result of the user request. Although a lot of computing may take place “behind the scene” the user should experience a near-instantane-ous answer to the request.

Example: In the following aspects of the present invention will be exemplified using a very simple set-up.

It should be noted that knowledge of the actual current strategy is not essential for the performance of the present invention—it could even be a ‘no strategy’ in which the patient just takes a fixed bolus each morning. The benchmarking algorithm provides a framework to compare new algorithms (e.g. algorithm X) with the method that the patient is already using. It is enough to know the current strategy's output glucose values and thus its treatment outcomes. The output of the patient's current strategy in combination with the algorithm X and its output is enough to run the benchmarking.

Algorithm X is a bolus calculator with this formula:

$${Ins}_{a} = \frac{CHO}{CIR} + \frac{{CGM}_{premeal} - CG {M}_{target}}{ISF} ,$$

wherein:

insₐ=the computed bolus size (IU) using algorithm X

CHO=carbohydrates

CIR=carbohydrate to insulin ratio

ISF=insulin sensitivity factor

CGMₚᵣₑₘₑₐₗ=glucose measured at pre-meal-time using continuous glucose monitoring

CGM_{target}=the target glucose level

The physiological model (PM) of the effect of bolus insulin on interstitial glucose:

$${IG}_{{Ins}_{a}} ( s ) = \frac{{K}_{2}}{{( 1 + {T}_{2} s )}^{2} s} {Ins}_{a} ( s ) ,$$

wherein:

K₂=−40mg/dl/IU

T₂=50 min

The above physiological model is an example of a simple linear model in Laplace domain. The input of the model is the bolus insulin dose, and the model output is IGᵢₙₛ which is the change in Interstitial Glucose (IG) caused by bolus insulin. IGᵢₙₛ has negative values, because it is a deviation variable reflecting the reduction of interstitial glucose due to insulin.

The output of the model in time domain is IGᵢₙₛ_{ₐ}(t) (see FIG. 3), which is the inverse Laplace transform of IGᵢₙₛ_{ₐ}(s) and it is computed as:

$${IG}_{{Ins}_{a}} ( t ) = {L}^{- 1} ( \frac{{K}_{2}}{{( 1 + {T}_{2} s )}^{2} s} {Ins}_{a} ( s ) )$$

IGᵢₙₛ(t) is a time series.

In the second arm, Ins in FIG. 3 is the bolus insulin taken by the patient and it is determined (computed) using the current strategy. Using the same physiological model for Ins, the (time domain) modelled deviation change in IG due to Ins is computed as:

$${IG}_{Ins} ( t ) = {L}^{- 1} ( \frac{{K}_{2}}{{( 1 + {T}_{2} s )}^{2} s} Ins ( s ) )$$

In the following example a deviation analysis for Algorithm X and a current strategy using the model above will be shown, see FIG. 3.

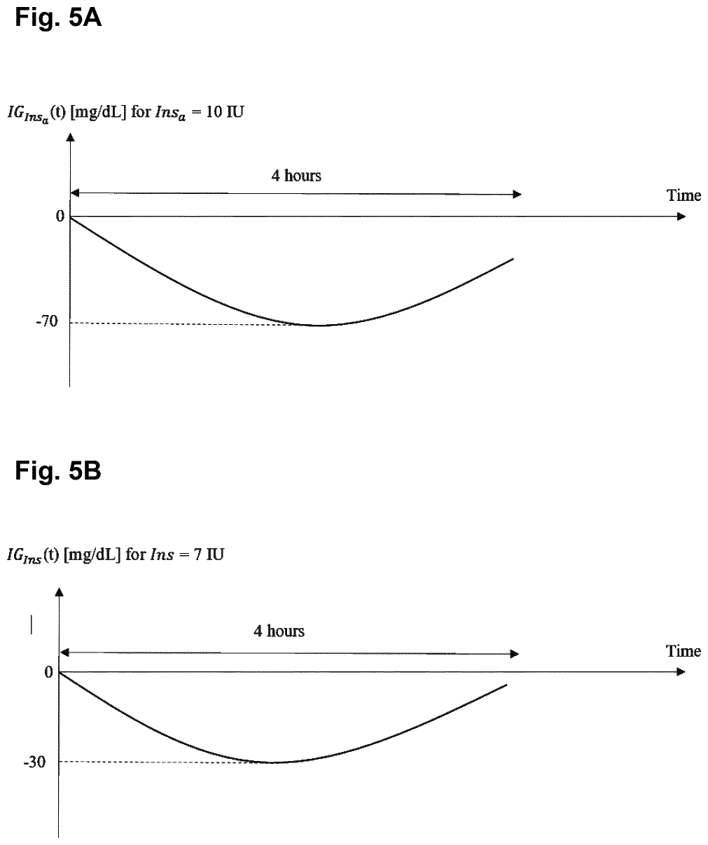

If it is assumed that for day 1 algorithm X computed a morning bolus dose of insₐ=10 units and the current strategy computed a morning bolus dose of Ins=8 units for the same breakfast meal at day 1. Using the model in the previous section, the 4-hour postprandial time series of IGᵢₙₛ_{ₐ}(t) and IGᵢₙₛ_{ₐ} (t) look like the graph shown in FIG. 5A. The bolus is injected at time=0. The model output for the current strategy is shown in FIG. 5B.

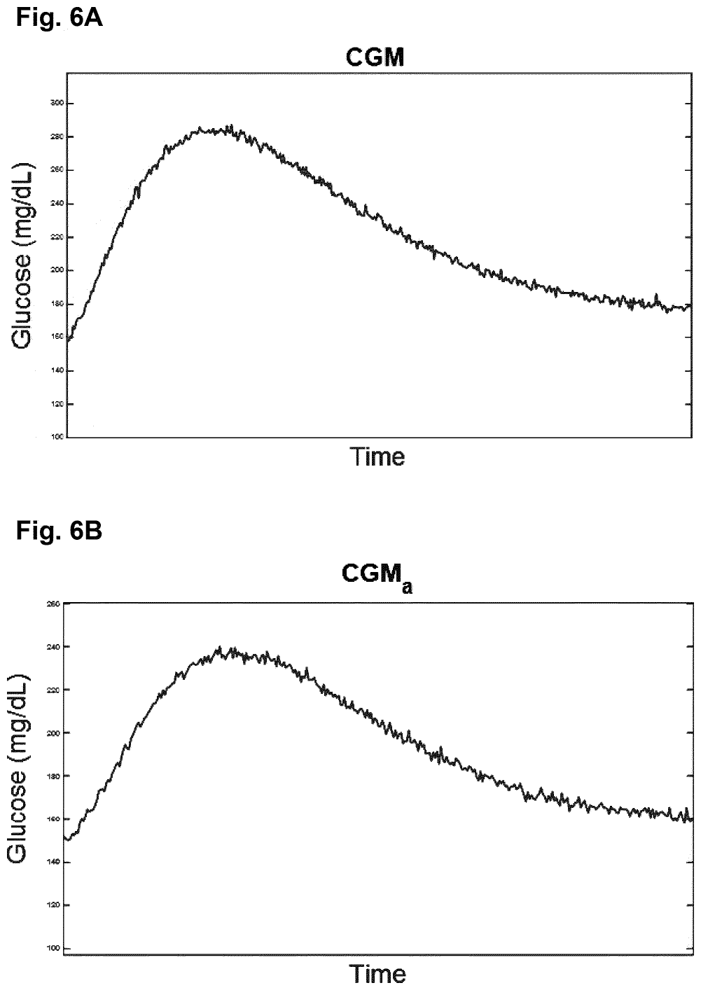

The measured CGM (see FIG. 3) for the 4-hour postprandial interval has the time series shown in FIG. 6A.

CGMₐ (see FIG. 3) is the simulated 4-hour postprandial glucose profile for Algorithm X using the deviation analysis in FIG. 3, and it is computed as CGMₐ(t)=CGM(t)+IGᵢₙₛ_{ₐ}(t)−IG_{Ins}(t). CGMₐ(t) has the time series shape shown in FIG. 6B.

The benchmarking algorithm computes the treatment outcomes, [X₁, X₂, X₃], from CGM(t) and CGMₐ(t) which correspond to the bolus insulin computed using the current strategy and algorithm X respectively. The subsequent application of a statistical test will be shown and explained in greater detail in the below statistical calculation example in which three treatment outcomes for two treatment methods are compared.

| Methods compare | Description |
|---|---|
| 1. Current | The current method that the patient |
| strategy | uses to compute a bolus dose |
| | for breakfast |
| 2. Algorithm X | A bolus calculator algorithm |

| Treatment outcomes | Description |
|---|---|
| X₁: Time in range % | The percentage of time that the CGM |
| | signal is in the target glycemic range |
| | in the 3 hours post-meal interval |
| X₂: Time in | The percentage of time that the CGM |
| hypoglycemia % | signal is in hypoglycemic range |
| | in the 3 hours post-meal interval |
| X₃: Glycemic | GA is measured by coefficient of |
| variability (GA) % | variation (CV) for the 3 hours |
| | post-meal interval |

For each new dose event, treatment outcomes [X₁, X₂, X₃] generated for each dosing method (current and algorithm X) are used to calculate a weighted performance score, S=exp(λ₁X₁+λ₂X₂+λ₃X₃), that penalises poor outcomes and rewards desirable outcomes.

Time in range % is desired outcome and time in hypoglycemia % and glycemic variability are poor outcomes. λ₁=1, and λ₂=λ₃=31 1. For every dose event the weighted performance score is computed as follows.

For the Current strategy: S_{current}=exp(1×X₁−1×X₂−1×X₃),

For algorithm X: Sₓ=exp(1×X₁−1×X₂−1×X₃),

| | | Current strategy | | | | | Algorithm X | | | | | |
|---|---|---|---|---|---|---|---|---|---|---|---|---|
| | | Bolus | | | | | Bolus | | | | | Performance |
| | | dose | | | | | dose | | | | | ratio |
| Dose event | Contextual data | size (IU) | X₁ | X₂ | X₃ | S_{current} | size (IU) | X₁ | X₂ | X₃ | Sₓ | $\frac{{S}_{X}}{{S}_{Current}}$ |
| Day 1 | Morning | 6 | 67% | 3% | 15% | 1.63 | 8 | 77% | 4% | 21% | 1.68 | 1.03 |
| | bolus | | | | | | | | | | | |
| Day 2 | Morning | 5 | 52% | 1% | 10% | 1.51 | 6 | 60% | 1% | 8% | 1.66 | 1.11 |
| | bolus | | | | | | | | | | | |
| Day 3 | Morning | 4 | 40% | 0% | 5% | 1.42 | 4 | 44% | 0% | 4% | 1.49 | 1.05 |
| | bolus | | | | | | | | | | | |
| Day 4 | Morning | 8 | 80% | 5% | 20% | 1.73 | 10 | 85% | 6% | 23% | 1.75 | 1.01 |
| | bolus | | | | | | | | | | | |
| Day 5 | Morning | 4 | 34% | 0% | 4% | 1.35 | 5 | 56% | 1% | 7% | 1.62 | 1.2 |
| | bolus | | | | | | | | | | | |
| Day 6 | Morning | 6 | 55% | 1% | 11% | 1.54 | 6 | 58% | 1% | 9% | 1.62 | 1.05 |
| | bolus | | | | | | | | | | | |
| Day 7 | Morning | 7 | 65% | 2% | 8% | 1.73 | 9 | 78% | 5% | 24% | 1.63 | 0.94 |
| | bolus | | | | | | | | | | | |
| Day 8 | Morning | 5 | 36% | 0% | 6% | 1.35 | 5 | 46% | 0% | 5% | 1.51 | 1.12 |
| | bolus | | | | | | | | | | | |
| Day 9 | Morning | 6 | 70% | 4% | 17% | 1.63 | 7 | 79% | 5% | 21% | 1.70 | 1.04 |
| | bolus | | | | | | | | | | | |

Ratio t-Test for the Performance Ratio:

Null hypothesis:

$$\frac{{S}_{X}}{{S}_{C u r r e n t}} = 1$$

Alternative hypothesis:

$$\frac{{S}_{X}}{{S}_{Current}} \neq 1 ,$$

which means either

$$\frac{{S}_{X}}{{S}_{C u r r e n t}} > 1 OR \frac{{S}_{X}}{{S}_{C u r r e n t}} < 1 .$$

The patient continues with the current strategy in two cases:

1) The test does not reject the null hypothesis

2) The test rejects the null hypothesis (the alternative hypothesis is true) with

$$\frac{{S}_{X}}{{S}_{Current}} < 1 .$$

The patient switches to algorithm X in case:

The test rejects the null hypothesis (the alternative hypothesis is true) with

$$\frac{{S}_{X}}{{S}_{C u r r e n t}} > 1 .$$

Step 1 of the test: Transform all

$$\frac{{S}_{X}}{{S}_{C u r r e n t}}$$

values to their logarithm.

Step 2 of the test: A one-sample t-test on the

$$y = log ( \frac{{S}_{X}}{{S}_{Current}} )$$

is performed to see if the mean of y is equal to zero (null hypothesis) of if it is different from zero (alternative hypothesis).

Test results in MATLAB:

| Matlab command | [h, p, ci, stats] = ttest(y) |
|---|---|
| p-value | 0.0389 < 0.05 |
| 95% ci (Confidence interval of | [0.0037 0.1106] |
| the mean of y) | |
| Mean of y | 0.0572 |
| df (Degrees of freedom of the | 8 |
| test = # of observations −1) | |
| t-statistics | 2.4665 |

Results show that p-value<0.05 indicating that the null hypothesis is rejected, which means that the mean of y is different from 0. This also indicates that the ratio,

$$\frac{{S}_{X}}{{S}_{Current}} ,$$

is different from 1. The ci of

$$\frac{{S}_{X}}{{S}_{Current}}$$

is the antilogarithm of the ci of the mean of y, which is [1.0037 1.1169]. The lower and upper bounds of the confidence interval of

$$\frac{{S}_{X}}{{S}_{C u r r e n t}}$$

are greater than 1 and do not include 1, which means that statistically Sₓ>S_{current}. Therefore, the patient switches to algo-rithm X for calculating the morning boluses.

Contextual labels can also be applied towards recognising specific sets of conditions under which performance is trusted. For example, if a subset of performance scores corresponding to morning events results in significantly superior performance of the algorithm compared to the user, e.g. as shown in the above example, the algorithm could be allowed to provide advice under these same conditions. Where it is not possible to compare conclusively with the available data, the user may be asked for additional input. This could include e.g. a meal size estimation. These contextual labels (identifiers) can be gathered from devices already included in the benchmarking algorithm setup (e.g. timestamps from a connected insulin pen), the user's mobile phone, as well as from other connected devices such as wearable biosensors (e.g. information about physical activity from an activity tracker).

When a patient would like to start using a dose guidance tool (algorithm/app) in which selected dose guidance tools are benchmarked against the user's current dosing strategy to guide se-lection of an appropriate dose guidance tool and ensure its superiority over the user's current strategy, e.g. official ADA guidelines, the following set-up may be applied:

At start-up alternate doses suggested by the dose guidance tools are not communicated to the user while benchmarking runs in the background. When after a period of time, e.g. 2 weeks, benchmarking has shown a new dose strategy to be safe, efficacious, and superior to the user's current dose strategy, it can be enabled and run, i.e. dose suggestions based on the better-performing alternative DGA are communicated to user. When a change in dose strategy is required, e.g. due to a change in the underlying physiological model upon which the dose guidance tool was previously benchmarked, the dose guidance tool is disabled and “safe mode” is activated until the dose guidance tool is enabled for the updated user model. Safe mode could be the user's previous strategy, or a conservative dosing strategy such as official ADA guidelines.

The present invention can be implemented as a computer program product that comprises a computer program mechanism embedded in a non-transitory computer readable storage me-dium and be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

For example, as an alternative way of estimating response to algorithm dose than deviation analysis, a ‘net effect’ analysis may be used. In this method it is assumed that blood glucose variations come from some ‘known’ inputs and some ‘unknown’ inputs. The known inputs are the physiological model of insulin-glucose transfer function which we have specified for that specific patients. The unknown inputs are all sources of variations that cannot be directly modelled, but their effect on blood glucose using deconvolution or moving horizon estimation can be estimated.

dG1/dt=f(insulin that patient actually took,t)+w(t), in which

f is the individualized identified insulin model (known input). W(t) is the effect of unknown inputs, e.g., stress, illness, meal, physical activity, insulin model imperfection, etc. For the application in the present context, meal is also an unknown input because we do not want to bother patients to count their carbohydrate and give it to the algorithm for a meal model.

When the net effect, i.e., w″(t) is estimated, then glucose variation for the case if the patient would take the insulin dose advised by the algorithm is estimated.

dG2/dt=f(insulin that algorithm suggests,t)+w^{∧}(t)

Then the treatment outcomes of G1 and G2 are compared using CUSUM test. Now the desired treatment outcomes can be extracted and the performance of the patient's decision with the algorithm advice can be compared.

An alternative to ratio t-test can be any change detection or event detection technique. The event that we want to detect is the outperformance of the algorithm over the patient's own decisions. One option is cumulative sum change detection (CUSUM) since it is optimal for detections that are not abrupt but gradual.

1. A computing system for providing medication dose guidance recommendations for a query subject to treat diabetes mellitus, wherein the system comprises one or more processors and a memory, the memory comprising:
instructions that, when executed by the one or more processors, perform a method of evaluating and benchmarking one or more alternative dose guidance algorithms (DGAs) against a current DGA, the instructions comprising the steps of:
obtaining a first data set, comprising a plurality of glucose measurements of the query subject taken over a time course and thereby establish a blood glucose history (BGH), each respective glucose measurement in the plurality of glucose measurements comprising:
(i) a blood glucose (BG) value, and
(ii) a corresponding blood glucose timestamp representing when in the time course the respective glucose measurement was made,
obtaining a second data set, comprising an insulin dose event history (IH) of the query subject, the IH comprising at least one dose event during all or a portion of the time course, each dose event of the at least one dose event comprising:
(i) an insulin dose amount, and
(ii) a corresponding dose event timestamp representing when in the time course the respective dose event occurred,
obtaining one or more alternative DGAs adapted to calculate an alternative dose recommendation based at least on BGH,
obtaining a physiological model (PM) for the query subject adapted for modelling a BG response based on BGH and an amount of insulin injected at a given time,
corresponding to a recent dose event performed in accordance with the current DGA and resulting in a corresponding measured BG treatment outcome, for a given alternative DGA:
i) determining an alternative dose recommendation,
ii) utilizing the PM to calculate a corresponding alternative BG treatment outcome,
iii) comparing and benchmarking the alternative BG treatment outcome against the measured BG treatment outcome,
if the benchmarking for the given alternative DGA exceeds a given set of benchmarking criteria, then suggest/make the given alternative DGA substitute the current DGA.

2. The computing system as in claim 1, wherein for a given alternative DGA:
the step of comparing and benchmarking is performed for a plurality of alternative BG treatment outcomes against the corresponding measured BG treatment outcome for a plurality of dose events performed over a time course.

3. The computing system as in claim 2, wherein:
the steps of comparing, benchmarking and substituting are performed for a plurality of alternative BG treatment outcomes in accordance with an identifier representing a specific condition.

4. The computing system as in claim 3, wherein the specific condition is a specific event and/or a specific period of time.

5. The computing system as in claim 1, wherein:
the step of comparing and benchmarking one or more alternative BG treatment outcomes for one or more alternative DGAs is performed using a statistical test.

6. The computing system as in claim 1, wherein:
the step of comparing and benchmarking is performed for a plurality of DGAs.

7. The computing system as in claim 1, wherein the instructions comprise the further steps of:
for a given current dose recommendation:
(i) utilizing the PM to calculate a calculated BG treatment outcome for the dose recommendation, and
(ii) calculating a deviation BG outcome as the difference between the measured BG treatment outcome and the calculated BG treatment outcome,
for the corresponding alternative BG treatment outcome for a given alternative DGA, calculate a corrected alternative BG treatment outcome as the sum of the alternative BG treatment outcome and the deviation BG outcome,
wherein the corrected alternative BG treatment outcome is utilized in the comparing and benchmarking step.

8. The computing system as in claim 1, wherein a substituted current DGA becomes a new alternative DGA.

9. The computing system as in claim 1, wherein the DGAs are adapted for calculation of a bolus amount of fast-acting insulin.

10. The computing system as in claim 1, wherein the DGAs are adapted for calculation of a dose recommendation for a long- or ultra-long-acting insulin, each DGA representing a given level of aggressiveness in a dose titration regimen.

11. The computing system as in claim 1, wherein the instructions comprise the further step of:
determining a current dose recommendation utilizing the current DGA.

12. The computing system as in claim 1, comprising a smartphone with a display, the display being controlled to display suggested substitutions of DGAs.
